(19) United States
(12) Patent Application Publication
(10) Pub. No.: US 2025/0025097 A1
(43) Pub. Date: Jan. 23, 2025

(54) METHOD FOR MONITORING TREPOPNEA IN HEART FAILURE PATIENTS

(71) Applicant: Cardiac Pacemakers, Inc., St. Paul, MN (US)

(72) Inventors: John D. Hatlestad, Maplewood, MN (US); Pramodsingh Hirasingh Thakur, Woodbury, MN (US); Bin Mi, Arden Hills, MN (US); Jonathan Bennett Shute, Eagan, MN (US)

(21) Appl. No.: 18/775,575

(22) Filed: Jul. 17, 2024

Related U.S. Application Data
(60) Provisional application No. 63/528,187, filed on Jul. 21, 2023.

(51) Int. Cl. A61B 5/00; A61B 5/107; A61B 5/11

(52) U.S. Cl. CPC A61B 5/4818; A61B 5/1071; A61B 5/1116; A61B 5/6802; A61B 5/686; A61B 5/7275

(57) ABSTRACT

An ambulatory medical device includes a multi-axis posture sensor and processing circuitry. The multi-axis posture sensor is configured to provide an electrical posture sensor output representative of alignment of respective first, second, and third non-parallel axes of the ambulatory medical device with the gravitational field of the earth. The processing circuitry is configured to determine that the subject avoids lying on their left side using the posture sensor output, and compute a metric predictive of one or both of orthopnea and trepopnea in response to determining that the subject avoids lying on their left side.

### CLAIM OF PRIORITY

This application claims the benefit of U.S. Provisional Application No. 63/528,187 filed on Jul. 21, 2023, which is hereby incorporated by reference in its entirety.

### TECHNICAL FIELD

This document relates generally to cardiac rhythm management systems and particularly, but not by way of limitation, to methods, systems, and devices for automatic monitoring of trepopnea of a patient using an ambulatory medical device.

### BACKGROUND

Ambulatory medical devices (AMDs) include implantable medical devices (IMDs), insertable cardiac monitors (ICMs), and wearable cardiac monitors. Some examples of IMDs include cardiac function management (CFM) devices such as implantable pacemakers, implantable cardioverter defibrillators (ICDs), cardiac resynchronization therapy devices (CRTs), and devices that include a combination of such capabilities. The devices can be used to treat patients or subjects using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Other examples of IMDs include implantable diagnostic devices, implantable drug delivery systems, or implantable devices with neural stimulation capability.

Wearable cardiac monitors can include surface electrodes. The surface electrodes are arranged to provide one or both of monitoring surface electrocardiograms (ECGs) and delivering cardioverter and defibrillator shock therapy.

Some AMDs include one or more sensors to monitor different physiologic aspects of the patient. Sensing of patient posture can provide information related to a patient's condition or disease. For example, a patient with congestive heart failure (CHF) may tend to sleep in an elevated position as their condition worsens. Monitoring of the orientation of the patient may provide useful information to a caregiver in diagnosing the condition of the patient.

### SUMMARY

Device-based monitoring of heart failure patients avoiding lying on their left side can provide information regarding the patient's condition. The information and analysis can be performed outside of a clinical setting.

Example 1 includes subject matter (such as a method of operating an ambulatory medical device, or AMD) comprising determining that the subject avoids lying on their left side using a multi-axis posture sensor of the AMD and computing a metric predictive of one or both of orthopnea and trepopnea in response to determining that the subject avoids lying on their left side. The posture sensor is configured to provide an electrical posture sensor output representative of alignment of respective first, second, and third non-parallel axes of the AMD with a gravitational field of the earth.

In Example 2, the subject matter of Example 1 optionally includes trending, by the AMD, an amount of time that the subject is lying on the left side, and generating an indication of heart failure status of the subject using the trend.

In Example 3, the subject matter of one or both of Examples 1 and 2 optionally includes determining an elevation angle of the subject using a calibrated posture sensor output, determining a side angle of the subject using the calibrated posture sensor output, and trending an amount of time that the determined elevation and side angles indicate that the subject is on their left side.

In Example 4, the subject matter of Example 3 optionally includes generating a status of the one or both of orthopnea and trepopnea of the subject using the determined elevation angle and determined side angle.

In Example 5, the subject matter of one or any combination of Examples 1-4 optionally includes determining the direction of a current gravity vector relative to axes of the posture sensor, computing an angle change between the current gravity vector and a direction of a previously measured gravity vector relative to the axes of the posture sensor, and computing multiple angle changes and determining that the subject is on their left side using the determined angle changes.

In Example 6, the subject matter of Example 5 optionally includes determining the angle change using a dot-product of the current gravity vector and the previously measured gravity vector.

In Example 7, the subject matter of one or both of Examples 5 and 6 optionally includes determining that the subject is on their left side using a predetermined percentage of largest angle changes of the multiple angle changes.

In Example 8, the subject matter of one or any combination of Examples 1-7 optionally includes calibrating an output of the multi-axis posture sensor to an orientation of the AMD relative to an orientation of the subject, and determining that the subject is stationary and lying on their left side using the calibrated output of the multi-axis posture sensor.

Example 9 includes subject matter (such as an AMD) or can optionally be combined with one or any combination of Examples 1-8 to include such subject matter, comprising a multi-axis posture sensor configured to provide an electrical posture sensor output representative of alignment of respective first, second, and third non-parallel axes of the AMD with the gravitational field of the earth, and processing circuitry communicatively coupled to the multi-axis posture sensor. The processing circuitry is configured to determine that the subject avoids lying on their left side using the posture sensor output, and compute a metric predictive of one or both of orthopnea and trepopnea in response to determining that the subject avoids lying on their left side.

In Example 10, the subject matter of Example 9 optionally includes processing circuitry configured to detect that the subject is stationary using a calibrated posture sensor output, determine that the subject avoids lying on their left side using the calibrated posture sensor output, and compute a metric predictive of one or both of orthopnea and trepopnea in response to determining that the subject avoids lying on their left side.

In Example 11, the subject matter of one or both of Examples 10 and 11 optionally includes processing circuitry configured to compute an elevation angle of the subject using the posture sensor output, compute a side angle of the subject using the posture sensor output, and compute, as the metric, an amount of time that the elevation angle of the subject is within a predetermined range of a flat elevation and the side angle indicates that the subject is on their left side.

In Example 12, the subject matter of Example 11 optionally includes processing circuitry configured to produce a status of one or both of orthopnea and trepopnea of the subject using the determined elevation angle and determined side angle.

In Example 13, the subject matter of one or nay combination of Examples 9-12 optionally includes processing circuitry configured to compute the direction of a first gravity vector relative to axes of the posture sensor, compute the direction of a second gravity vector relative to the axes of the posture sensor, compute an angle change between the second gravity vector and the first gravity vector, and compute successive angle changes between the second gravity vector and the first gravity vector determine that the subject avoids lying on their left side using the computed successive angle changes.

In Example 14, the subject matter of Example 13 optionally includes processing circuitry configured to compute a dot-product of the second gravity vector and the first gravity vector to compute the angle change between the second gravity vector and the first gravity vector.

In Example 15, the subject matter of one or both of Examples 13 and 14 optionally includes processing circuitry configured to compute multiple gravity vectors and compute multiple angle changes between the gravity vectors, identify a predetermined percentile of computed angle changes that have a highest value of computed angle changes, compute a mean value of the computed angle changes, and determine that the subject avoids lying on their left side based on the mean value of the computed angle changes.

In Example 16, the subject matter of one or any combination of Examples 9-15 optionally includes a posture sensor that is a multi-axis accelerometer, and processing circuitry configured to calibrate an output of the multi-axis accelerometer to an orientation of the AMD relative to the subject, and determine that the subject is stationary and lying on their left side using the calibrated output of the multi-axis accelerometer.

In Example 17, the subject matter of one or any combination of Examples 9-16 optionally includes a communication circuit operatively coupled to the processing circuitry and configured to communicate information to a separate device, and processing circuitry configured to generate an indication of heart failure status of the subject using the amount of time that the subject is lying on their left side, and send the indication to the separate device.

Example 18 includes subject matter (such as a programming device for an AMD) or can optionally be combined with one or any combination of Examples 1-17 to include such subject matter, comprising a communication circuit configured to communicate information wirelessly with the AMD, a user interface, and a programming control circuit operatively coupled to the communication circuit and user interface. The programming control circuit is configured to receive orientation information of a subject from the AMD, compute a metric indicative of the subject avoiding lying on their left side using the orientation information, and produce an indication of heart failure status of the subject according to a comparison of the metric to a trepopnea detection threshold.

In Example 19, the subject matter of Example 18 optionally includes a programming control circuit configured to receive a trend of side angle information of the subject from the AMD, and produce the indication of heart failure status using the trend of side angle information.

In Example 20, the subject matter of Example 18 optionally includes a programming control circuit configured to receive a trend of orientation angle change information from the AMD, wherein the angle change information includes angle changes computed between consecutive gravity vectors measured by the AMD; and produce the indication of heart failure status using the trend of angle change information.

These nonlimiting Examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example Cardiac Rhythm Management (CRM) system.

FIG. 2 illustrates an example of an ambulatory medical device (AMD) and an environment in which it operates.

FIG. 3 is a block diagram of the electronic circuits of an AMD.

FIG. 4 is a block diagram of an external device that communicates information with an AMD.

FIGS. 5A-5C illustrate examples of patient orientation.

FIG. 6 is a flow diagram of a method of operating a CRM system.

FIG. 7 shows an example of a trend of side angle information of the subject.

FIG. 8 shows an example of a coordinate system for a patient's body.

FIG. 9 shows an example of a device coordinate system of an AMD.

FIG. 10 shows another example of a device coordinate system skewed from body coordinates of the patient using the device.

FIGS. 11A-11B show an example of a trend of side angle measurements by a calibrated multi-axis accelerometer and a trend of Maximum Gravity Vector Angle Change (Max GVAC) measured using an uncalibrated multi-axis accelerometer.

FIGS. 12A and 12B are illustrations of patient elevation angles.

FIG. 13 shows graphs of the mean of Max GVAC values for patients.

### DETAILED DESCRIPTION

Ambulatory medical devices (AMDs) can be used to provide cardiac pacing therapy to a patient or other subject. AMDs can include, or be configured to receive physiologic information from, one or more sensors located within, on, or proximate to a body of a patient. Physiologic information of the patient can include, among other things, respiration information (e.g., a respiratory rate, a respiration volume (tidal volume), acceleration information (e.g., cardiac vibration information, heart sound information, endocardial acceleration information, activity information, posture information, etc.); impedance information; cardiac electrical information; pressure information; plethysmograph information; chemical information; temperature information; or other physiologic information of the patient.

Posture sensing with a medical device can improve patient monitoring. Some examples of a posture sensor include a multi-axis accelerometer and a tilt switch. With a posture sensor, a medical device can detect whether a patient is in an upright position, a supine position, a prone position, on his or her left or right side, or if the patient is in a tilted position. For patients with heart failure, posture sensing allows monitoring of orthopnea; a condition where the patient can breathe easily only when in an upright or mostly upright posture (e.g., standing or sitting with the torso upright). Posture sensing allows monitoring of trepopnea; a condition where the patient avoids lying down on their left side. Patients with congestive heart failure may avoid sleeping on their left side. Monitoring trepopnea of a patient with an AMD can provide information regarding the heart failure status of the patient.

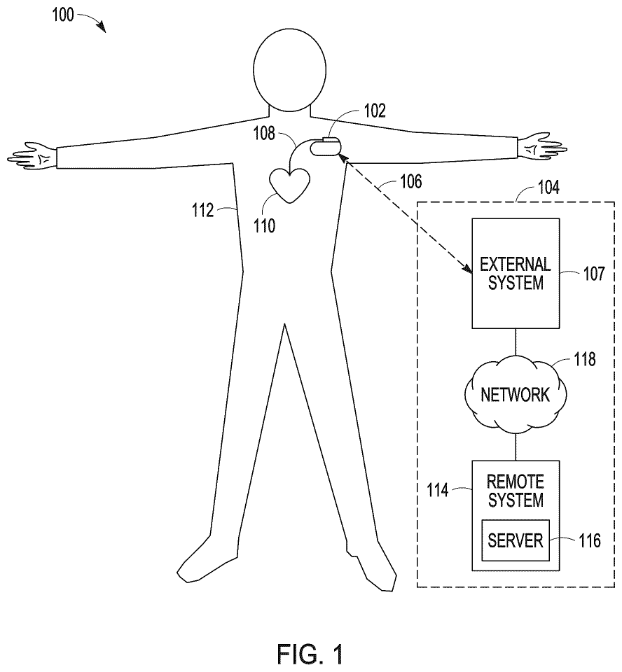

FIG. 1 illustrates portions of an example of a CRM system 100 and portions of an environment in which the CRM system 100 can be used. The CRM system 100 can include an AMD 102 that is implantable, an external system 104, and a communication link such as a telemetry link 106. The AMD 102 can include an electronic unit coupled by a cardiac lead 108, or additional leads, to a heart 110 of a subject 112. Examples of the AMD 102 can include, but are not limited to, pacemakers, pacemaker/defibrillators, cardiac resynchronization devices, cardiac remodeling control devices, and cardiac monitors. In an example, the AMD 102 can be configured to monitor health of the heart 110 and determine one or more abnormalities associated with the heart 110. The AMD 102 can take a necessary action, such as stimulating one or more portions of the heart 110 through the lead 108, to treat the one or more abnormalities.

In an example, the external system 104 can include an external device 107 configured to communicate bi-directionally with the AMD 102 such as through the wireless telemetry link 106. For example, the external device 107 can include a programmer to program the AMD 102 to provide one or more therapies to the heart 110. In an example, the external device 107 can program the AMD 102 to detect presence of a conduction block in the heart 110 and prevent dyssynchronous contraction of the heart 110 by providing a cardiac resynchronization therapy (CRT) to the heart 110.

In an example, the external device 107 can be configured to transmit data to the AMD 102 through the telemetry link 106. Examples of such transmitted data can include programming instructions for the AMD 102 to acquire physiological data, perform at least one self-diagnostic test (such as for a device operational status), or deliver at least one therapy or any other data. In an example, the AMD 102 can be configured to transmit data to the external device 107 through the telemetry link 106. This transmitted data can include real-time physiological data acquired by the AMD 102 or stored in the AMD 102, therapy history data, an operational status of the AMD 102 (e.g., battery status or lead impedance), and the like. The telemetry link 106 can include an inductive telemetry link or a radio-frequency telemetry link.

In an example, the external device 107 can be a part of a CRM system that can include other devices such as a remote system 114 for remotely programming the AMD 102. In an example, the remote system 114 can include a server 116 that can communicate with the external device 107 through a telecommunication network 118 such as to access the AMD 102 to remotely monitor the health of the heart 110 or adjust parameters associated with the one or more therapies.

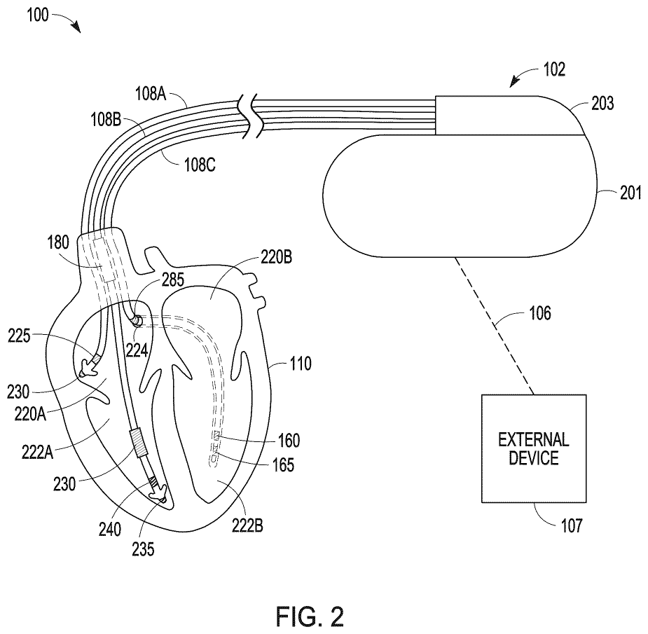

FIG. 2 illustrates a CRM system 100 that includes an AMD 102 that is implantable and is electrically coupled to a heart 110, such as through one or more leads 108A, 108B, 108C, coupled to the AMD 102 through one or more lead ports in a header 203 of the AMD 102. In an example, the AMD 102 can include an antenna, such as in the header 203, configured to enable communication with an external system and one or more electronic circuits (e.g., an assessment circuit, etc.) in a hermetically sealed housing (CAN) 201. The AMD 102 illustrates an example ambulatory medical device (or a medical device system) as described herein. The system 100 also includes an AMD programmer or other external device 107 that communicates wireless signals with the AMD 102 using telemetry link 106.

Cardiac leads 108A, 108B, 108C include a proximal end that is coupled to AMD 102 and a distal end, coupled by electrical contacts or “electrodes” to one or more portions of a heart 110. The electrodes typically deliver cardioversion, defibrillation, pacing, or resynchronization therapy, or combinations thereof to at least one chamber of the heart 110. The electrodes can be electrically coupled to sense amplifiers to sense electrical cardiac signals.

Heart 110 includes a right atrium 220A, a left atrium 220B, a right ventricle 222A, a left ventricle 222B, and a coronary sinus 224 extending from right atrium 220A. Right atrial (RA) lead 108A includes electrodes (electrical contacts, such as ring electrode 225 and tip electrode 230) disposed in the right atrium 220A of heart 110 for sensing signals, or delivering pacing therapy, or both, to the atrium 100A.

Right ventricular (RV) lead 108B includes one or more electrodes, such as tip electrode 235 and ring electrode 240, for sensing signals, delivering pacing therapy, or both sensing signals and delivering pacing therapy. Lead 108B optionally also includes additional electrodes, such as for delivering atrial cardioversion, atrial defibrillation, ventricular cardioversion, ventricular defibrillation, or combinations thereof to heart 110. Defibrillation electrodes typically have larger surface areas than pacing electrodes to handle the larger energies involved in defibrillation. Lead 108B optionally provides resynchronization therapy to the heart 110. Resynchronization therapy is typically delivered to the ventricles to better synchronize the timing of depolarizations between ventricles.

Lead 108B can include RV defibrillation coil electrode 275 located proximal to tip and ring electrodes 235, 240 for placement in a right ventricle, and a second defibrillation coil electrode 280 located proximal to the RV defibrillation coil 275, tip electrode 235, and ring electrode 240 for placement in the superior vena cava (SVC). In some examples, high-energy shock therapy is delivered from the r RV coil 275 to the second or SVC coil 280. In some examples, the SVC coil 280 is electrically tied to an electrode formed on the CAN 201. This improves defibrillation by delivering current from the RV coil 275 more uniformly over the ventricular myocardium. In some examples, the therapy is delivered from the RV coil 275 only to the electrode formed on the CAN 201.

The AMD 102 can include a third cardiac lead 108C attached to the AMD 102 through the header 203. The third cardiac lead 108C includes ring electrodes 260 and 265 placed in a coronary vein lying epicardially on the left ventricle (LV) 222B via the coronary vein 216. The third cardiac lead 108C can include a ring electrode 285 positioned near the coronary sinus (CS) 224.

Note that although a specific arrangement of leads and electrodes are shown the illustration, the present methods and systems will work in a variety of configurations and with a variety of electrodes. Other forms of electrodes include meshes and patches which can be applied to portions of heart 110 or which can be implanted in other areas of the body to help “steer” electrical currents produced by AMD 102.

An AMD can be configured with a variety of electrode arrangements, including transvenous, endocardial, and epicardial electrodes (i.e., intrathoracic electrodes), and/or subcutaneous, non-intrathoracic electrodes, including can, header, and indifferent electrodes, and subcutaneous array or lead electrodes (i.e., non-intrathoracic electrodes).

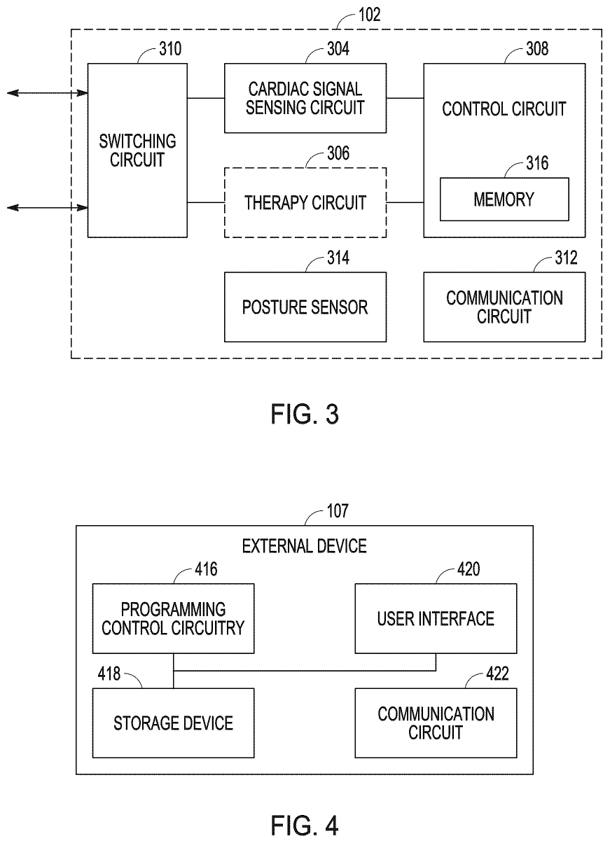

FIG. 3 is a block diagram of portions of electronic circuits of an AMD 102 that is implantable. The AMD 102 can be coupled to multiple implantable electrodes, such as the electrode arrangement described in the example of FIG. 2. The AMD 102 includes a cardiac signal sensing circuit 304, a therapy circuit 306, a posture sensor 314, a switching circuit 310, a communication circuit 312, and a control circuit 308. The therapy circuit 306 provides electrical pacing stimulation energy to the heart of the patient when operatively connected to pacing electrodes of the system. The pacing electrodes can include any of the pacing electrodes in FIG. 2, such as electrodes configured placement in or near the RA, RV, LV, His Bundle, or left bundle branches, and an electrode of the CAN.

The cardiac signal sensing circuit 304 includes one or more sense amplifiers to sense one or both of a voltage signal or a current signal at the electrodes. Cardiac electrical information of the patient can be sensed using the cardiac signal sensing circuit 304. Timing metrics between different features in a sensed electrical signal (e.g., first and second cardiac features, etc.) can be determined, such as by the control circuit 308. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc. Far-field cardiac signals can be sensed using the electrode of the CAN 201.

The switching circuit 310 is to electrically couple different combinations of the electrodes to the therapy circuit 306 and the cardiac signal sensing circuit 304. The switching circuit 310 can configure any combination of the electrodes into a pacing vector to deliver cardiac pacing stimulation energy or configure any combination of the electrodes into a sensing vector to sense a cardiac signal.

The control circuit 308 may include a digital signal processor, application specific integrated circuit (ASIC), field programmable gate array (FPGA), microprocessor, or other type of processor, interpreting or executing instructions in software or firmware. In some examples, the control circuit 308 may include a state machine or sequencer that is implemented in hardware circuits. The control circuit 308 may include any combination of hardware, firmware, or software. The control circuit 308 includes one or more circuits to perform the functions described herein. A circuit may include software, hardware, firmware or any combination thereof. For example, the circuit may include instructions in software executing on the control circuit 308. Multiple functions may be performed by one or more circuits of the control circuit 308. The control circuit 308 uses the communication circuit 312 to communicate information wirelessly with a separate device.

FIG. 4 is a block diagram of portions of an example of an external device 107 (e.g., external device 107 of the CRM system 100 in FIG. 1) to communicate with the AMD 102 of FIG. 3. The external device 107 may be a programming device for the AMD 102. Programming device includes a storage device 418, a programming control circuit 416, a user interface 420, and a communication circuit 422. Programming control circuit 416 may be implemented using an application-specific integrated circuit (ASIC) constructed to perform one or more particular functions or a general-purpose circuit programmed to perform such functions. A general-purpose circuit can include, among other things, a microprocessor or a portion thereof, a microcontroller or portions thereof, and a programmable logic circuit or a portion thereof. The storage device 418 may be a memory integral to the programming control circuit 416, or a separate memory device. Communication circuit 422 communicates information wirelessly with the AMD 102 using near-field inductive wireless signals or far-field radio-frequency signals. The programming device can be used to program pacing therapy parameters and other information in the AMD 102.

Returning to FIG. 3, orientation information of the patient can be determined using the output of the posture sensor 314. Using the output from the posture sensor 314, the control circuit 308 can determine elevation angle and side angle. Elevation angle θ_{E} is the between the torso of the patient and the horizontal plane.

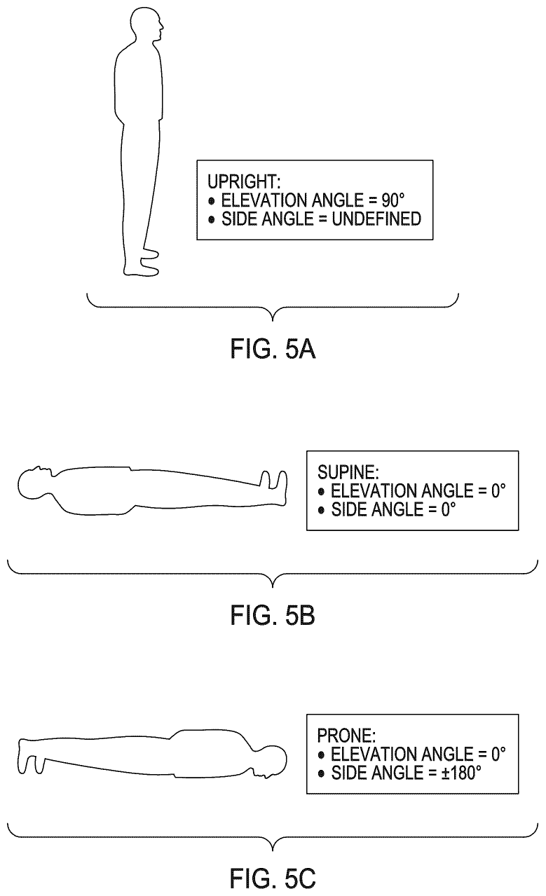

FIGS. 5A-5C illustrate examples of patient orientation. In FIG. 5A the patient is standing upright and the elevation angle is 90°. In FIGS. 5B and 5C, the patient is lying down and the elevation angle is 0°. When the patient is lying down, the side angle φ indicates which side the patient is lying on. In FIG. 5B, the patient is shown supine, and the elevation angle is 0° and the side angle is also 0°. In FIG. 5C, the patient is shown prone, and the elevation angle is 0° and the side angle is 180°. When the patient is lying on their right side the elevation angle is 0° and the side angle is close to +90°. When the patient is lying on their left side the elevation angle is 0° and the side angle is close to −90°.

By monitoring the amount of time that the patient is lying on their left side when lying down, the AMD 102 can detect when the patient is avoiding lying on their left side. Trending this information regarding the orientation of the patient can provide information on the progression of the patient's disease.

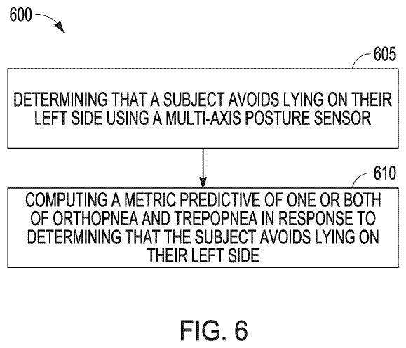

FIG. 6 is a flow diagram of a method 600 of operating a CRM system that includes an AMD 102 and an external device 107. The AMD 102 may be implanted in a patient or worn by a patient. The AMD 102 includes a posture sensor 314. The posture sensor 314 provides an electrical posture sensor output that is representative of alignment of respective first, second, and third non-parallel axes of the AMD 102 with the gravitational field of the earth.

The AMD 102 detects that the subject is stationary using the output of the posture sensor 314. For instance, the posture sensor may be a multi-axis accelerometer, and the control circuit 308 of the AMD 102 may detect that the subject is stationary when the acceleration of the patient is below a threshold acceleration, or that the output of the accelerometer is constant and not changing with time (e.g., due to earth's gravitational field). In some examples, the control circuit 308 may detect that the subject is stationary when the mean of the acceleration of the patient is below a threshold mean acceleration value for a predetermined amount of time.

At block 605, when the subject is determined to be stationary, the AMD 102 determines that the subject avoids lying on their left side using the posture sensor output. The control circuit 308 may compute the elevation angle θ_{E} of the subject and the side angle φ of the subject. The orientation information of the patient and the amount time the patient spends in one or more of the detected orientations can be recorded by the control circuit 308 by storing the information in memory 316 of the AMD 102. At block 610, the AMD 102 computes a metric predictive of orthopnea and trepopnea in response to determining that the subject avoids lying on their left side. The computed metric can be a trend of the amount of time that the patient is lying on their left side. The trend can be used to generate an indication of heart failure status of the patient.

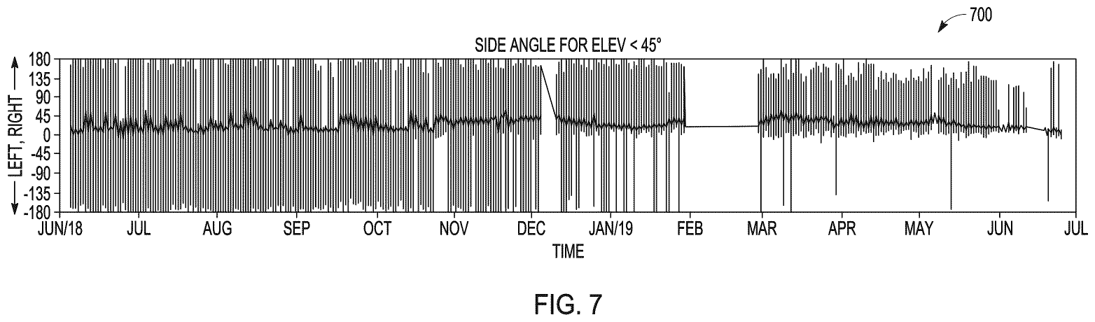

FIG. 7 shows an example of a trend 700 of side angle information of the patient. In this example, the trend begins with a patient lying on both of their left and right sides, then gradually stops lying on the left side by the end of the trend. To create the trend 700, the control circuit 308 periodically (e.g., every ten minutes) records the side angle of the patient when the AMD 102 determines that the patient is stationary and that the elevation angle θ_{E} of the patient is less than 45°. The control circuit 308 can be programmed to record the side angle at other elevation angles or range of elevation angles. In some examples, the left side orientation of the patient is identified when the computed side angle is −90° or near −90°. In some examples, the left side orientation of the patient is identified using a range of side angles (e.g., −75° to −115°).

In some examples, the AMD 102 sends the side angle information and the time spent in the side angle to the external device 107. The external device 107 computes the amount of time that the patient is lying on their left side and may produce the trend 700 for display to a physician. The external device may generate an indication of heart failure status of the patient using the orientation information and the time information. The indication may be presented on the display of the external device 107. The heart failure status may be an indication of one or both of trepopnea and orthopnea of the patient. The indication of heart failure status can be presented with the trend 700 of side angle information. In the example of FIG. 7, the indication of heart failure status may be an alert of worsening heart failure status of the patient presented with the trend 700.

In some examples, the control circuit 308 of the AMD 102 determines when the patient is on their left side using the side angle information and computes the amount of time that the patient is lying on their left side. The AMD 102 may send an indication (e.g., a signal or message) of heart failure status to the external device 107 using the communication circuit 312.

A complication that can arise is that the AMD 102 and posture sensor 314 may not have an ideal orientation in or on the patient's body, and the orientation may change from patient to patient. In this case, the posture sensor 314 may have to be “body-calibrated.”

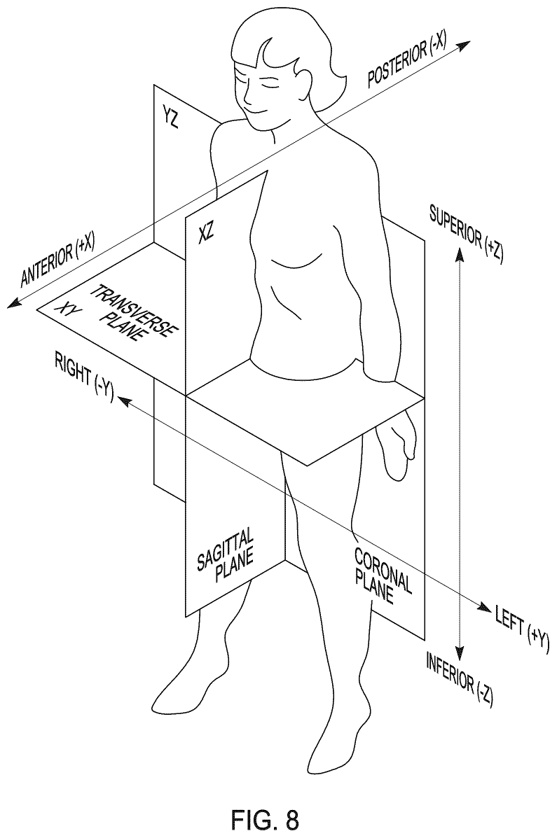

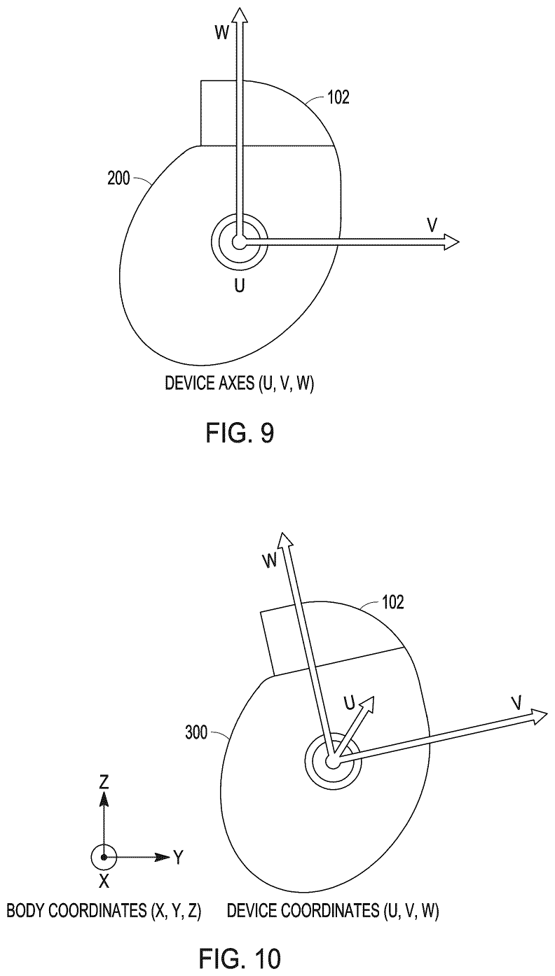

FIG. 8 shows a coordinate system for a patient's body. The body coordinate system uses the x, y, and z axes to describe the posterior to anterior direction, left to right direction, and inferior to superior direction respectively. FIG. 9 shows a device coordinate system using u, v, and w axes as shown. A desirable orientation of the AMD 102 can be in the coronal plane such that the device axes match the body axes (e.g., u↔x, v↔y, w↔z). However, a device orientation is not typically in an ideal orientation.

FIG. 10 shows the more typical case in which the device coordinates are skewed from the body coordinates. As discussed previously, this can be due to patient anatomy or due to movement of the AMD 102. A calibration procedure is used to determine the device orientation or to translate the device coordinates to body coordinates so that algorithms run by the medical device that use posture sensing can correct for the device orientation.

The calibration procedure involves determining the elements of a calibration matrix that can be used to calculate a coordinate transformation to transform the device coordinates to body coordinates. To determine the calibration matrix, the patient is placed in a first posture (e.g., an upright posture). The control circuit 308 measures the output of the posture sensor for two axes of the posture sensor. The patient is then placed in a second posture (e.g., a posture more supine than the upright posture) and the control circuit 308 again measures the output of the posture sensor for the same two axes of the posture sensor.

The measurements for the two postures are used to compute the calibration matrix. Once the calibration matrix is determined, the output of the posture sensor multiplied by the calibration matrix to generate outputs calibrated to body coordinates. The calibrated outputs of the posture sensor 314 can be used to determine the elevation angle and the side angle of the patient. An approach to calibration of a posture sensor can be found in Hatlestad et al., U.S. Pat. No. 10,328,267.

The manual steps of placing the patient in specified postures and prompting the AMD 102 to calibrate its posture sensor 314 uses time and effort by the clinicians, and sometime the posture sensor 314 of the AMD 102 is left uncalibrated. With an uncalibrated sensor, the AMD 102 cannot directly measure the elevation angle and side angle to assess the degree of orthopnea and trepopnea independently. However, the AMD 102 can perform an algorithm to compute a metric predictive of orthopnea and trepopnea using the axes of the posture sensor 314.

For instance, the posture sensor 314 may be a multi-axis accelerometer. Periodically throughout the day (e.g., every ten minutes) the control circuit 308 performs an acceleration measurement when the AMD 102 determines that the patient is stationary. The measurement is the direction of the gravity vector relative to the axes of the accelerometer. When a new gravity vector is measure, the control circuit 308 computes the angle change 48 between the new gravity vector and the direction of the previously measured gravity vector. This produces a value for the angle change Δθ between 0° and 180°. In some examples, the angle change Δθ is calculated using the normalized vector dot-product

$$Δ θ = {cos}^{- 1} ( \frac{a \cdot b}{( \vert a \vert \vert b \vert )} ) .$$

In some examples, the normalized vector dot-product without the inverse cosine can be trended, or

$$l = \frac{( a \cdot b )}{( ( \vert a \vert \vert b \vert ) )} .$$

This can reduce the energy consumed by the control circuit 308 for the computations.

A running maximum of consecutive angle changes between gravity vector measurements is stored over multiple days (e.g., 7-28 days). This measurement can be referred to as a Maximum Gravity Vector Angle Change (Max GVAC). For angle changes of about 180° while the subject is lying down it can be assumed that the patient rolled over from their left side to their right side, or vice versa. The angle changes are trended (by either the AMD 102, the external device 107, or a remote server 116). When the patient never lies on their left side, the Max GVAC is reduced.

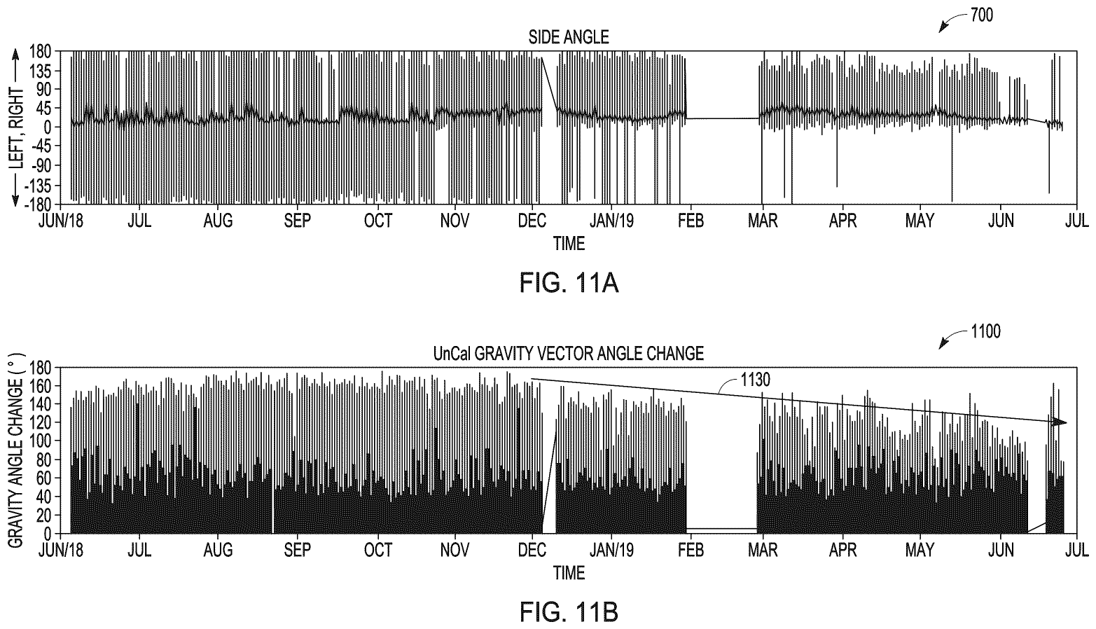

FIG. 11A shows the trend 700 of side angle measurements using a calibrated multi-axis accelerometer and FIG. 11B shows a trend 1100 of GVAC measured using an uncalibrated multi-axis accelerometer. As noted previously herein, the side angle trend 700 shows that the patient stops lying on the left side by the end of the trend and the trend is showing trepopnea of the patient. A comparison to the GVAC trend 1100 shows the maximum value of GVAC decreasing as the patient stops lying on their left side (as indicated by arrow 1130).

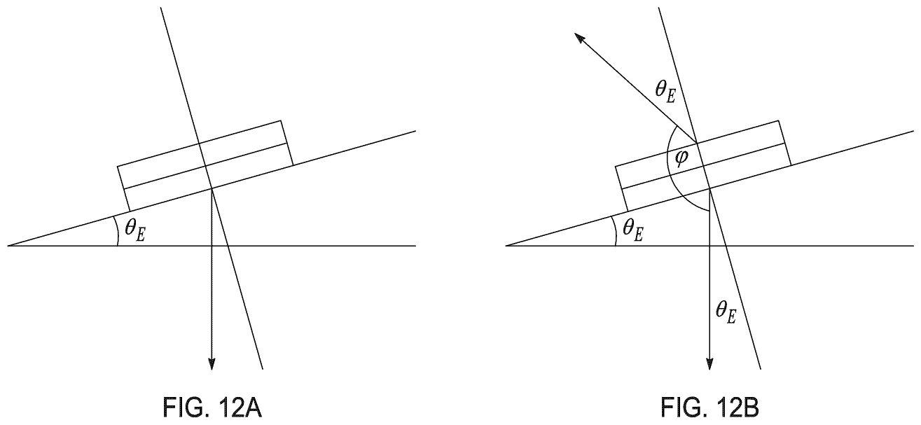

The maximum value of GVAC also reduces when the heart failure status of the patient worsens, and the patient begins sleeping with a raised elevation. FIG. 12A is illustration of an elevation angle θ_{E} of a rectangle representing the patient. FIG. 12B represents the patient after rolling over. If the rectangle was lying flat (θ_{E}=0), the GVAC between FIGS. 12A and 12B would be the side angle φ. But because the rectangle is elevated by the angle θ_{E}, the GVAC is reduced by 2θ_{E} (or GVAC=φ−2θ_{E}). This shows that the decrease in Max GVAC in the trend 1100 in FIG. 11 can also detect orthopnea of the subject.

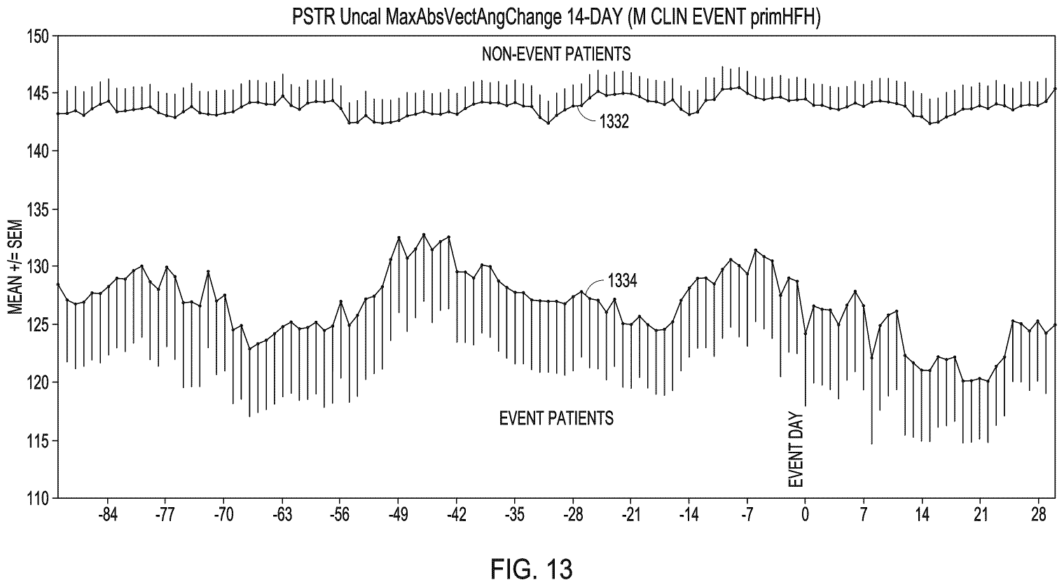

FIG. 13 is a graph 1332 of the mean of the Max GVAC values with an uncalibrated accelerometer of heart failure (HF) patients that did not experience a HF decompensation event and a graph 1334 of the mean Max GVAC values for heart failure patients who did experience a HF decompensation event. The graph shows that the Max GVAC values are lower for the more at-risk patients. Therefore, Max GVAC is an effective risk stratifier for heart failure hospitalizations.

In some examples, more angle changes other than just the maximum can be trended over time to detect trepopnea and orthopnea. For example, a percentile of the highest angle changes could be included in the trend instead of the just the maximum angle changes. In some examples, more than just consecutive angle changes can be used in the trend. For instance, all angle changes that occur within a predetermined time window (e.g., a one-hour time window or two-hour time window).

The detection of trepopnea and orthopnea may be improved by taking the Max GVAC measurements when the patient is asleep and not just stationary. The AMD 102 may include additional sensors (e.g., a heart rate sensor or a respiration rate sensor). The control circuit 308 can use the outputs of the posture sensor 314 and the additional sensors can be used to detect that the patient is sleeping.

The systems, methods and devices described herein provide device-based collection and analysis of orientation data of heart failure patients. The data is collected using a multi-axis posture sensor of a device worn by or implanted into a patient. The data can be analyzed by a CRM system to monitor status or progression of the patient's condition.

### Additional Description

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A method of operating an ambulatory medical device (AMD) wearable by a subject or implantable in the subject, the method comprising:
determining that the subject avoids lying on their left side using a multi-axis posture sensor of the AMD, wherein the posture sensor is configured to provide an electrical posture sensor output representative of alignment of respective first, second, and third non-parallel axes of the AMD with a gravitational field of the earth; and
computing a metric predictive of one or both of orthopnea and trepopnea in response to determining that the subject avoids lying on their left side.

2. The method of claim 1, wherein the computing the metric includes:
trending, by the AMD, an amount of time that the subject is lying on the left side; and
generating an indication of heart failure status of the subject using the trend.

3. The method of claim 1,
wherein the determining that the subject avoids lying on their left side includes:
determining an elevation angle of the subject using a calibrated posture sensor output;
determining a side angle of the subject using the calibrated posture sensor output; and
wherein the computing the metric includes trending an amount of time that the determined elevation and side angles indicate that the subject is on their left side.

4. The method of claim 3, including:
generating a status of the one or both of orthopnea and trepopnea of the subject using the determined elevation angle and determined side angle.

5. The method of claim 1, wherein the determining that the subject avoids lying on their left side includes:
determining the direction of a current gravity vector relative to axes of the posture sensor;
computing an angle change between the current gravity vector and a direction of a previously measured gravity vector relative to the axes of the posture sensor; and
computing multiple angle changes and determining that the subject is on their left side using the determined angle changes.

6. The method of claim 5, including determining the angle change using a dot-product of the current gravity vector and the previously measured gravity vector.

7. The method of claim 5, wherein the determining that the subject avoids lying on their left side includes determining that the subject is on their left side using a predetermined percentage of largest angle changes of the multiple angle changes.

8. The method of claim 1, including:
calibrating an output of the multi-axis posture sensor to an orientation of the AMD relative to an orientation of the subject; and
wherein the determining that the subject avoids lying on their left side includes determining that the subject is stationary and lying on their left side using the calibrated output of the multi-axis posture sensor.

9. An ambulatory medical device (AMD), the device comprising:
a multi-axis posture sensor configured to provide an electrical posture sensor output representative of alignment of respective first, second, and third non-parallel axes of the AMD with the gravitational field of the earth; and
processing circuitry communicatively coupled to the multi-axis posture sensor and configured to:
determine that the subject avoids lying on their left side using the posture sensor output; and
compute a metric predictive of one or both of orthopnea and trepopnea in response to determining that the subject avoids lying on their left side.

10. The device of claim 9, wherein the processing circuitry is configured to:
detect that the subject is stationary using a calibrated posture sensor output;
determine that the subject avoids lying on their left side using the calibrated posture sensor output; and
compute, as the metric, an amount of time that the subject is lying on their left side.

11. The device of claim 9, wherein the processing circuitry is configured to:
compute an elevation angle of the subject using the posture sensor output;
compute a side angle of the subject using the posture sensor output; and
compute, as the metric, an amount of time that the elevation angle of the subject is within a predetermined range of a flat elevation and the side angle indicates that the subject is on their left side.

12. The device of claim 11, wherein the processing circuitry is configured to produce a status of one or both of orthopnea and trepopnea of the subject using the determined elevation angle and determined side angle.

13. The device of claim 9, wherein the processing circuitry is configured to:
compute the direction of a first gravity vector relative to axes of the posture sensor;
compute the direction of a second gravity vector relative to the axes of the posture sensor;
compute an angle change between the second gravity vector and the first gravity vector; and
compute successive angle changes between the second gravity vector and the first gravity vector determine that the subject avoids lying on their left side using the computed successive angle changes.

14. The device of claim 13, wherein the processing circuitry is configured to:
compute a dot-product of the second gravity vector and the first gravity vector to compute the angle change between the second gravity vector and the first gravity vector.

15. The device of claim 13, wherein the processing circuitry is configured to:
compute multiple gravity vectors and compute multiple angle changes between the gravity vectors;
identify a predetermined percentile of computed angle changes that have a highest value of computed angle changes;
compute a mean value of the computed angle changes; and
determine that the subject avoids lying on their left side based on the mean value of the computed angle changes.

16. The device of claim 9,
wherein the posture sensor is a multi-axis accelerometer; and
wherein the processing circuitry is configured to:
calibrate an output of the multi-axis accelerometer to an orientation of the AMD relative to the subject; and
determine that the subject is stationary and lying on their left side using the calibrated output of the multi-axis accelerometer.

17. The device of claim 9, including:
a communication circuit operatively coupled to the processing circuitry and configured to communicate information to a separate device; and
wherein the processing circuitry is configured to:
generate an indication of heart failure status of the subject using the amount of time that the subject is lying on their left side; and
send the indication to the separate device.

18. A programming device for an ambulatory medical device (AMD), the programming device comprising:
a communication circuit configured to communicate information wirelessly with the AMD;
a user interface; and
a programming control circuit operatively coupled to the communication circuit and user interface; the programming control circuit configured to:
receive orientation information of a subject from the AMD;
compute a metric indicative of the subject avoiding lying on their left side using the orientation information; and
produce an indication of heart failure status of the subject according to a comparison of the metric to a trepopnea detection threshold.

19. The programming device of claim 18, wherein the programming control circuit is configured to:
receive a trend of side angle information of the subject from the AMD; and
produce the indication of heart failure status using the trend of side angle information.

20. The programming device of claim 18, wherein the programming control circuit is configured to:
receive a trend of orientation angle change information from the AMD, wherein the angle change information includes angle changes computed between consecutive gravity vectors measured by the AMD; and
produce the indication of heart failure status using the trend of angle change information.
